(19) United States
(12) Patent Application Publication
(10) Pub. No.: US 2025/0010052 A1
(43) Pub. Date: Jan. 9, 2025

(54) Soft shading needles for tattooing

(71) Applicant: Iuliia Penza, Hollywood, FL (US)

(72) Inventor: Iuliia Penza, Hollywood, FL (US)

(73) Assignee: Artem Penza, Hollywood, FL (US)

(21) Appl. No.: 18/591,439

(22) Filed: Feb. 29, 2024

Related U.S. Application Data
(60) Provisional application No. 63/525,136, filed on Jul. 5, 2023.

(51) Int. Cl. A61M 37/00

(52) U.S. Cl. CPC A61M 37/0076

(57) ABSTRACT

The invention is a set of single needles assembled into a certain unique shape (2 options). Thanks to which it is supposed to achieve the main goal-smooth shading with evenly scattered edges and a center over large areas. The uniqueness of the form lies in the absence of analogues and the expected effect of its use. The new needle shape should make the artist's work easier.

### CROSS-REFERENCE TO RELATED APPLICATIONS

an improved type of tattoo needle, which combines two types of existing tattoo needles (not for permanent makeup): magnum and shader.

### 3. STATEMENT REGARDING FEDERALLY SPONSORED RESEARCH OR DEVELOPMENT

N/A

### THE NAMES OF THE PARTIES TO A JOINT RESEARCH AGREEMENT

n/a

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

text file

### STATEMENT REGARDING PRIOR DISCLOSURES BY THE INVENTOR OR A JOINT INVENTOR

N/A

### BACKGROUND OF THE INVENTION

### Field of the Invention

tattoo industry, not for permanent makeup

### Description of Related Art Including Information Disclosed Under 37 CFR 1.97 and 1.98

(1) Any tattoo cartridge is a set of single needles collected in special form, which has a special mark when used and the effect of its use. The closest in structure and application now are magnum and shader needles.

(2) The existing round shader needle contains up to 24 single needles in the form of a circle FIG. 19. It is intended only for dense shading and thick lines. The needles are too close to each other, the edge of the needle is straight, the number of needles is not enough to make soft shading FIG. 19B, FIG. 19C, FIG. 19D

(3) Magnum needle consists of one or two rows of needles in the form of a strip FIG. 18. Magnum is used for shading, but with a lack of skill in using the edges and shading are sharp and uneven FIG. 18B, FIG. 18C, FIG. 18D.

(4) Tattoo needles for permanent makeup are compatible only with permanent makeup tattoo machines.

### BRIEF SUMMARY OF THE INVENTION

This is a new shape of a tattoo needle in two versions to create soft shading and diffuse edges in large and small areas, which will make it easier for beginners and tattoo artists with a low level of professionalism.

### BRIEF DESCRIPTION OF THE SEVERAL VIEWS OF THE DRAWING(S)

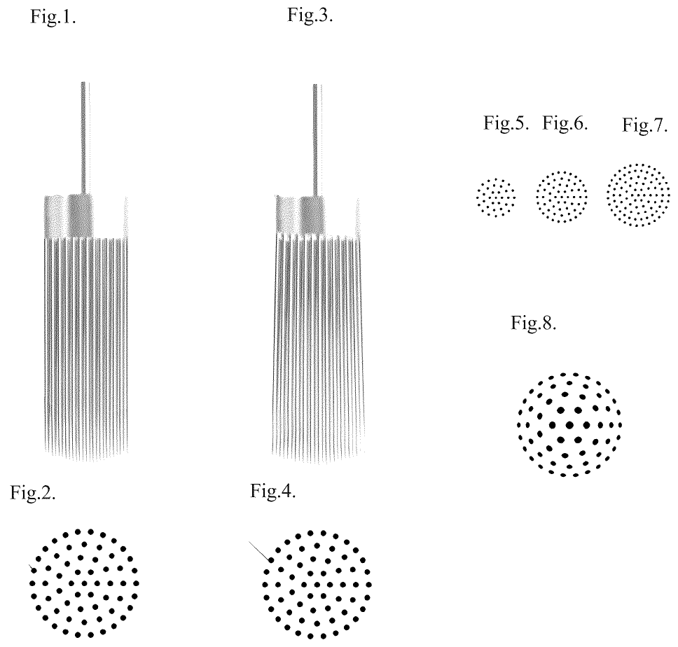

(1) FIG. 1—option 1 of a new form-Soft shader, 60 single needles, shows common view of the straight needle forming a hemisphere at the end without cartridge from the side. Needles are perpendicular to the surface and parallel to each other, forming a cylinder shape.

(2) FIG. 2 is bottom view-needles' mark, it has an identical taper. Each dot is a trace from single needle. All dots are the same size because the needles have the same sharpening.

(3) FIG. 3—option 2 of a new form-Fluffy soft shader, 60 single needles, shows common view of the fluffy needle forming a hemisphere at the end without cartridge from the side. Needles are at an angel to the surface and to each other, forming a cone shape.

(4) FIG. 4 is bottom view-needles' mark, it has an identical taper. Each dot is a trace from single needle. All dots are the same size because the needles have the same sharpening.

(5) FIG. 5 shows possible sizes 34 needles.

(6) FIG. 6 shows possible sizes 60 needles.

(7) FIG. 7 shows possible sizes 91 needles.

(8) FIG. 8—possible combined taper (short taper in a center, medium taper in a middle, long taper on the edge).

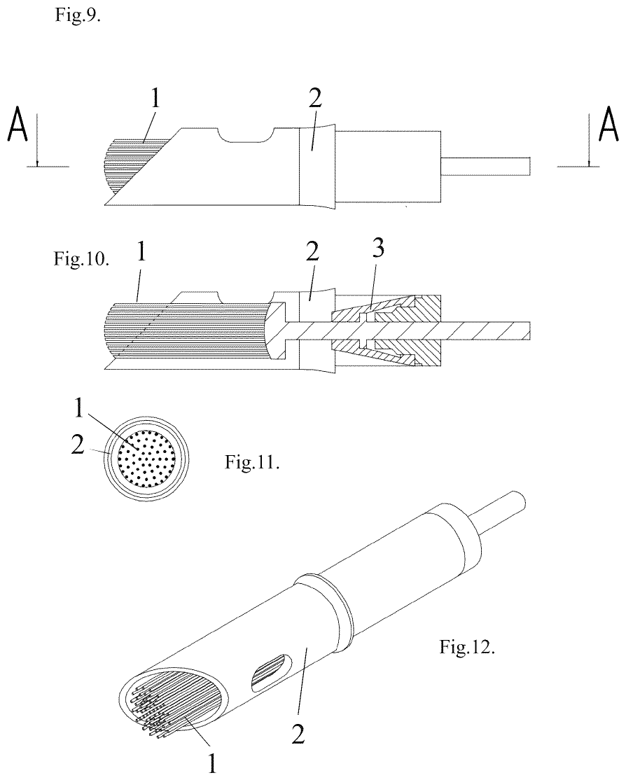

(9) FIG. 9.—common view of option 1 of a tattoo needle shape assembled with cartridge on a scale of 2:1. 1—Single needles, 2—cartridge.

(10) FIG. 10—sectional view. 1—Single needles, 2—cartridge, 3—membrane system.

(11) FIG. 11—side view. 1—Single needles, 2—cartridge.

(12) FIGS. 12—3d view. 1—Single needles, 2—cartridge.

(13) FIG. 13—common view of option 2 of a tattoo needle shape assembled with cartridge on a scale of 2:1. 1—Single needles, 2—cartridge.

(14) FIG. 14—sectional view. 1—Single needles, 2—cartridge, 3—membrane system.

(15) FIG. 15—side view, 1—single needles, 2—cartridge.

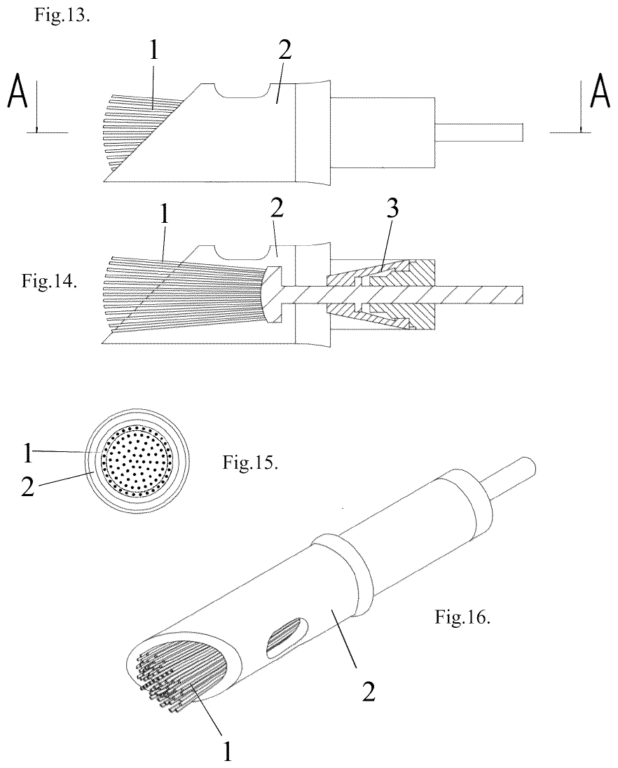

(16) FIGS. 16—3d view, 1—single needles, 2—cartridge.

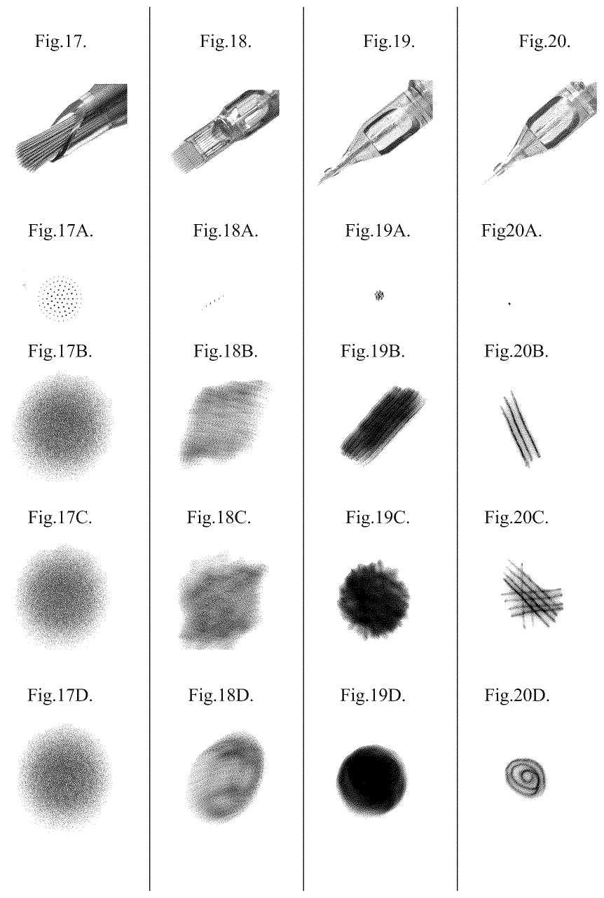

(17) FIG. 17—prototype of a new needle form “Soft fluffy shader” for crating soft and subtle shading of large areas and diffuse edges. FIG. 17A—single mark. FIG. 17B—multiple marks in straight direction have soft center and edges. FIG. 17C—multiple marks in chaotic direction have soft center and edges. FIG. 17D—multiple marks in circle direction have soft center and edges.

(18) FIG. 18—Existing needle “Magnum” for shading and dense painting of large areas. FIG. 18A—single mark. FIG. 18B—multiple marks in straight direction have soft center and uneven sharp edge. FIG. 18C—multiple marks in chaotic direction have uneven and sharp center and edge. FIG. 18D—multiple marks in circle direction have uneven and sharp center and soft round edge.

(19) FIG. 19—existing needle “Shader” for dense painting of small areas and thick lines. FIG. 19A—single mark. FIG. 19B—multiple marks in straight direction have dense coloring and sharp edge. FIG. 19C—multiple marks in straight direction have dense coloring and sharp edge. FIG. 19D—multiple marks in straight direction have dense coloring and sharp edge.

(20) FIG. 20—existing needle “Liner” for thin lines. FIG. 20A—single mark. FIG. 20B—multiple marks in straight direction represent lines. FIG. 20C—multiple marks in chaotic direction represent lines. FIG. 20D—multiple marks in circle represent lines.

### DETAILED DESCRIPTION OF THE INVENTION

At the moment, there are several forms for working with tattoos: magnum, shader, liner. The liner is intended for creating thin lines, the shader is for densely shading small areas and creating thick lines and the magnum is for creating soft shading and painting large areas. A comparison of needles by appearance, purpose, and marks from needle use are presented in FIG. 17 (FIG. 17A, FIG. 17B, FIG. 17C, FIG. 17D), FIG. 18 (FIG. 18A, FIG. 18B, FIG. 18C, FIG. 18D). FIG. 19 (FIG. 19A, FIG. 19B, FIG. 19C, FIG. 19D), FIG. 20 (FIG. 20A, FIG. 20B, FIG. 20C, FIG. 20D).

The main problem in realistic work that most tattoo artists face is creating soft shading with a smoothly dissipating edge in large open areas. This mainly applies to black and white portrait works, monochromatic semi-transparent backgrounds, clouds, and the like. To achieve the desired effect from a tattoo artist, high professionalism is required.

Currently, magnum needles of various sizes are used for this. The needle leaves a mark in the form of a strip. The faster the speed of the hand movement, the greater the distance between the stripes. If the tattoo artist turns the needle or changes the direction of movement, the mark from the needle will become crisscrossed and chaotic FIG. 18C. This gives the appearance of dirty and sloppy shading.

Many tattoo artists, especially beginners, do not know how to use magnum needles. The new needle shape should make the artist's work easier. The new needle shape is designed to create soft shading and smooth transitions between shades, mainly for portrait work, regardless of the direction of needle FIG. 17B, FIG. 17C, FIG. 17D. The new needle shape in two versions should eliminate the disadvantages of the magnum.

The single needles of the first needle option 1 illustrated on FIG. 1, FIG. 9, FIG. 10, FIG. 11, FIG. 12, are collected in a bunch and positioned strictly perpendicular to the surface and strictly parallel to each other forming a cylindrical shape, the edge of the needle is rounded. As well as the needles of the second option 1, illustrated on FIG. 3, FIG. 13, FIG. 14, FIG. 15, FIG. 16, expand relative to each other, i.e. at a slight angle to the surface to give a scattering boundary, the edge of the needle is rounded forming a cone form to enhance the effect of the scattering edge. A needle mark of both options in the form of a circle of dots, FIG. 2, FIG. 4. Drawings of new shapes soft shader 60 and fluffy soft shader 91 with approximate dimensions are shown in FIG. 9, FIG. 10, FIG. 11, FIG. 12 and FIG. 13, FIG. 14, FIG. 15, FIG. 16 on a scale of 2:1. The needle shape is similar to the shader shape, but it has a larger number of needles, the needles are located at a distance from each other and has an end in the form of a hemisphere. The greater the number of needles, the larger the area that can be painted and the smoother the shading should be. The possible size 34 needles is shown on FIG. 5. 60 needles-on FIGS. 6, and 91 needles-on FIG. 7. The smaller the distance between the needles, the denser the shading should be and vice versa. Too small a distance between single needles will bring the new needle shape closer to the existing shader, which will make the coloring dense and will not allow the shading to be light and translucent. The skin's resistance to puncture will also increase. In one bunch of needles, there can be different sharpening to create different densities of dyeing in one needle stroke. For example, in the center of the bundle there may be needles with a short sharpening, then with a medium sharpening and with a long sharpening FIG. 8. This way they can create a denser center and a more diffuse edge. The necessary distance between single needles, edge radius, sharpening of needles, the number of single needles and their location will be determined experimentally. The position of the needle and the direction of movement of the tattoo artist's hand should not affect the mark of the needle. In any position, the needle should leave multiply circles of dots and soft edges FIG. 17B, FIG. 17C, FIG. 17D. It will avoid «dirty» shading and uneven edges FIG. 18B, FIG. 18C, FIG. 18D.

To create a full-fledged tattoo cartridge with a new shape, it is supposed to use an adapted cartridge with an existing membrane system inside 3, illustrated in FIG. 10 and FIG. 14. An adapted cartridge implies an expanded capsule body 2 where needles are located, illustrated in FIG. 9, FIG. 10, FIG. 12 and FIG. 13, FIG. 14, FIG. 16.

1. The method of assembling the tattoo needle shape described in option 1 of the specification.

2. The method of assembling the tattoo needle shape described in option 2 of the specification.

3. According to point 1 the needles are perpendicular to surface and parallel to each other.

4. According to point 1 the needles have different lengths, forming a hemisphere.

5. According to point 1 the needles have identical sharpening or different.

6. According to point 1 the needles are spaced apart from each other, at least the thickness of one single needle.

7. According to point 1 the needles are collected in a bundle in the form of a cylinder.

8. According to point 7 cylindrical shape consists of different numbers of needles

9. According to point 7 cylindrical shape consists of needles in chaotic pattern.

10. According to point 2 the needles are at an angle to the surface and relative to each other

11. According to point 2 the needles have different lengths, forming a hemisphere.

12. According to point 2 the needles have identical sharpening or different

13. According to point 2 the needles are spaced apart from each other, at least the thickness of one single needle.

14. According to point 2 the needles are collected in a bundle in the form of a cone.

15. According to point 14 conical shape consists of different numbers of needles

16. According to point 14 conical shape consists of needles in chaotic pattern.
